(19) United States
(12) Patent Application Publication
(10) Pub. No.: US 2014/0127327 A1
(43) Pub. Date: May 8, 2014

(54) PRESERVED TOPICAL FORMULATIONS WITH IMPROVED ANTIMICROBIAL ACTIVITY

(71) Applicant: Allergan, Inc., Irvine, CA (US)

(72) Inventors: Richard S. Graham, Irvine, CA (US); Walter L. Tien, Irvine, CA (US)

(73) Assignee: Allergan, Inc., Irvine, CA (US)

(21) Appl. No.: 14/073,414

(22) Filed: Nov. 6, 2013

Related U.S. Application Data
(60) Provisional application No. 61/723,884, filed on Nov. 8, 2012.

(51) Int. Cl. A01N 59/00; A61K 31/568; A61K 31/573; A61K 31/498; A61K 31/165; A61K 38/13

(52) U.S. Cl. CPC A01N 59/00; A61K 31/165; A61K 38/13; A61K 31/573; A61K 31/498; A61K 31/568
USPC 424/661

(57) ABSTRACT

A preservative consisting of a stabilized oxychloro complex and a polyol having from 3 to 6 carbons is disclosed herein. A composition comprising a stabilized chlorine dioxide. and a polyol having from 3 to 6 carbons is also disclosed herein.

### CROSS REFERENCE TO RELATED APPLICATIONS

This application claims the benefit of U.S. Provisional Application Ser. No. 61/723,884, filed on Nov. 8, 2012, which is incorporated herein by reference herein in its entirety.

### DESCRIPTION OF THE INVENTION

A preservative is useful for preventing microbial growth in multi-use ophthalmic preparations. Oxidizing preservatives such as stabilized oxychloro complexes, including stabilized chlorine dioxide, and the like, are often less irritating that other preservatives such as benzalkonium chloride.

A preservative consisting of a stabilized oxychloro complex and a polyol having from 3 to 6 carbons is disclosed herein. The polyol is shown herein to improve the preservative efficacy of the stabilized oxychloro complex.

A composition comprising a stabilized chlorine dioxide. and a polyol having from 3 to 6 carbons is also disclosed herein.

These compositions are useful for treating various conditions of the eye, including dry eye and other conditions, and may be dispensed in a multi-use form. The use depends upon the particular composition and upon any therapeutically active agent used. Use of a therapeutically active agent in the composition is not required.

The preservative may be a single composition. Alternatively, the two components may be added separately to a composition to form a preservative in situ.

A stabilized oxychloro complex is a stabilized mixture of chlorine oxides of potentially varied oxidation states. For example, a stabilized oxychloro complex may comprise one or more of hypochlorites, chlorites, chlorates, or perchlorates, either individually or in combination. Examples of chlorite include stabilized chlorine dioxide, metal chlorites, such as alkali metal and alkaline earth metal chlorites, and the like and mixtures thereof. For, example technical grade (or USP grade) sodium chlorite is useful. The exact chemical composition of many chlorites, for example, stabilized chlorine dioxide, is not completely understood. The manufacture or production of certain chlorites is described in McNicholas U.S. Pat. No. 3,278,447, which is incorporated in its entirety herein by reference. Specific examples of useful stabilized chlorine dioxide products include that sold under the trademark Dura Klor by Rio Linda Chemical Company, Inc., and that sold under the trademark Anthium Dioxide by International Dioxide, Inc. An especially useful stabilized chlorine dioxide is a product sold under the trademark Purite® by Allergan, Inc.

In one embodiment, the concentration of Purite® is at least about 25 ppm.

In another embodiment, the concentration of Purite® is at least about 50 ppm.

In another embodiment, the concentration of Purite® is at least about 75 ppm.

In one embodiment, the concentration of Purite® is no more than about 100 ppm.

In another embodiment, the concentration of Purite® is no more than about 125 ppm.

In another embodiment, the concentration of Purite® is no more than about 150 ppm.

In another embodiment, the concentration of Purite® is no more than about 200 ppm.

In another embodiment, the concentration of Purite® is no more than about 300 ppm.

In another embodiment, the concentration of Purite® is no more than about 1000 ppm.

In another embodiment, the concentration of Purite® is from about 25 ppm to about 1000 ppm.

In another embodiment, the concentration of Purite® is from about 25 ppm to about 200 ppm.

In another embodiment, the concentration of Purite® is from about 50 ppm to about 100 ppm.

A polyol is a compound consisting of carbon, hydrogen, and at least two hydroxyl (i.e. OH) moieties. Examples include glycerin, C₂₋₆ sugars, propylene glycol, and the like.

In one embodiment the polyol is glycerin.

In another embodiment, the concentration of glycerin is at least about 0.001%.

In another embodiment, the concentration of glycerin is no more than about 3%.

In another embodiment, the concentration of glycerin is no more than about 2.6%.

In another embodiment, the concentration of glycerin is no more than about 2.2%.

In another embodiment, the concentration of glycerin is no more than about 1%.

In another embodiment, the concentration of glycerin is no more than about 0.3%.

In another embodiment, the concentration of glycerin is no more than about 0.2%.

In another embodiment, the concentration of glycerin is from about 0.001% to about 3%.

In another embodiment, the concentration of glycerin is from about 0.001% to about 2.6%.

In another embodiment, the concentration of glycerin is from about 0.001% to about 2.2%.

In another embodiment, the concentration of glycerin is from about 0.001% to about 1%.

In another embodiment, the concentration of glycerin is from about 0.001% to about 0.3%.

In another embodiment, the concentration of glycerin is from about 0.001% to about 0.2%.

In another embodiment, the concentration of glycerin is from about 0.2% to about 0.5%.

In one embodiment, the weight ratio of stabilized chlorine dioxide to polyol is at least about 0.000001.

In another embodiment, the weight ratio of stabilized chlorine dioxide to polyol is at least about 0.003.

In another embodiment, the weight ratio of stabilized chlorine dioxide to polyol is at least about 0.004.

In another embodiment, the weight ratio of stabilized chlorine dioxide to polyol is at least about 0.005.

In another embodiment, the weight ratio of stabilized chlorine dioxide to polyol is no more than about 0.01.

In another embodiment, the weight ratio of stabilized chlorine dioxide to polyol is no more than about 0.02.

In another embodiment, the weight ratio of stabilized chlorine dioxide to polyol is no more than about 0.05.

In another embodiment, the weight ratio of stabilized chlorine dioxide to polyol is no more than about 0.2.

In one embodiment the weight ratio of stabilized chlorine dioxide to polyol is from about 0.003 to about 0.05.

In another embodiment the weight ratio of stabilized chlorine dioxide to polyol is from about 0.004 to about 0.02.

In another embodiment the weight ratio of stabilized chlorine dioxide to polyol is from about 0.005 to about 0.01.

In another embodiment the weight ratio of stabilized chlorine dioxide to polyol is from about 0.01 to about 0.05.

In another embodiment the weight ratio of stabilized chlorine dioxide to polyol is from about 0.01 to about 0.02.

In another embodiment the weight ratio of stabilized chlorine dioxide to polyol is from about 0.02 to about 0.05.

In another embodiment, the weight ratio of stabilized chlorine dioxide to polyol is from about 0.003 to about 0.05.

In another embodiment, the weight ratio of stabilized chlorine dioxide to polyol is from about 0.000001 to about 0.2.

In another embodiment, the stabilized oxychloro complex is at least about 0.0001% of the preservative.

In one embodiment, the stabilized oxychloro complex is at least about 0.3% of the preservative.

In one embodiment, the stabilized oxychloro complex is at least about 0.4% of the preservative.

In one embodiment, the stabilized oxychloro complex is at least about 0.5% of the preservative.

In one embodiment, the stabilized oxychloro complex is no more than about 1% of the preservative.

In one embodiment, the stabilized oxychloro complex is no more than about 2% of the preservative.

In one embodiment, the stabilized oxychloro complex is no more than about 5% of the preservative.

In one embodiment, the stabilized oxychloro complex is no more than about 20% of the preservative.

In another embodiment, the stabilized oxychloro complex is from about 1% to about 2% of the preservative.

In another embodiment, the stabilized oxychloro complex is from about 2% to about 5% of the preservative.

In another embodiment, the stabilized oxychloro complex is from about 0.0001% to about 20% of the preservative.

In one embodiment, the stabilized oxychloro complex is from about 0.3% to about 5% of the preservative.

In another embodiment, the stabilized oxychloro complex is from about 1% to about 5% of the preservative.

In another embodiment, the stabilized oxychloro complex is from about 0.4% to about 2% of the preservative.

In another embodiment, the stabilized oxychloro complex is from about 0.5% to about 1% of the preservative.

In ophthalmic compositions, a chelating agent may be used to enhance preservative effectiveness. Suitable chelating agents are those known in the art, and, while not intending to be limiting, edetate (EDTA) salts like edetate disodium, edetate calcium disodium, edetate sodium, edetate trisodium, and edetate dipotassium are examples of useful chelating agents.

Buffers are well known by those skilled in the art and some examples of useful buffers are acetate, borate, carbonate, citrate, and phosphate buffers.

One embodiment has from about 0.50%to about 1.50% borate.

Another embodiment has about 1% borate.

A tonicity agent may be used to control the tonicity of the solution. The tonicity may be hypertonic, isotonic, or hypotonic, depending upon the particular need. Tonicity agents include, but are not limited to glycerin, propylene glycol, mannitol, sorbitol, glucose, sodium chloride, and other electrolytes.

In one embodiment, the composition is a hypotonic aqueous liquid. Hypotonic aqueous liquids have a tonicity which is below isotonic. If glycerin is used also as a tonicity agent, an isotonic aqueous has about 2.6% glycerin. So a hypotonic liquid may have, for example, from about 2.2% to just under about 2.6% glycerin.

One embodiment comprises about 2.20% glycerin.

Another embodiment comprises about 2.60% glycerin.

In one embodiment, the composition is a hypertonic aqueous liquid. Hypertonic aqueous liquids have a tonicity which is above isotonic. Thus, a hypertonic liquid may have, for example, from just over about 2.6% glycerin to about 3% glycerin.

Another embodiment comprises about 3.00% glycerin.

In one embodiment, the composition is an emulsion. Emulsions may be particularly useful for use in conjunction with a lipophilic therapeutically active agent. Some examples of lipophilic therapeutically active agents include, but are not limited to:
Prostaglandins—such as latanoprost, travoprost, unoprostone isopropyl, and the like;
Prostamides—such as bimatoprost and the like; and
Steroids—including estrogens; glucocorticoids; progestins; mineralocorticoids; corticosteroids, such as cortisone, hydrocortisone, prednisone, prednisolone, methylprednisone, triamcinolone, fluoromethalone, dexamethasone, medrysone, betamethasone, loteprednol, fluocinolone, flumethasone, or mometasone, and the like; androgens, such as testosterone, methyltestosterone, danazol, and the like.

In another embodiment, the composition is a solution. Solutions may be useful in conjunction with a water soluble therapeutically active agent. Some examples of useful water soluble therapeutically active agents include timolol, timolol maleate, and brimonidine.

Other useful therapeutically active agents include, but are not limited to, retinoids, prostaglandins, tyrosine kinase inhibitors, adrenoreceptor agonists or antagonists, dopaminergic agonists, cholinergic agonists, carbonic anhydrase inhibitors, guanylate cyclase activators, cannabinoids, endothelin, adenosine agonists, and neuroprotectants; analgesics/antipyretics such as aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, and meprobamate; antibiotics such as neomycin, streptomycin, chloramphenicol, cephalosporin, ampicillin, penicillin, and tetracycline; antidepressants such as nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, and protriptyline hydrochloride; antidiabetics such as biguanides, hormones, and sulfonylurea derivatives; antihypertensive agents such as propanolol, propafenone, oxyprenolol, Nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, and reserpine; anti-inflammatories such as indomethacin, naproxen, ibuprofen, ramifenazone, piroxicam, cortisone, dexamethasone, fluazacort, hydrocortisone, prednisolone, and prednisone; antineoplastics such as adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, and piposulfan; antianxiety agents such as lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and dantrolene; immunosuppressive agents such as cyclosporine, azathioprine, mizoribine, and tacrolimus; antimigraine agents such as ergotamine tartrate, propanolol hydrochloride, isometheptene mucate, and dichloralphenazone; antianginal agents such as beta-adrenergic blockers, nifedipine, diltiazem hydrochloride nitrates, nitroglycerin, isosorbide dinitrate, pentaerythritol tetranitrate, erythrityl and tetranitrate; antipsychotic agents such as haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, and prochlorperazine; antimanic agents such as lithium carbonate; antiarrhythmics such as bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, and lidocaine hydrochloride; antiarthritic agents such as phenylbutazone, sulindac, penicillamine, salsalate, piroxicam, azathioprine, indomethacin, meclofenamate sodium, gold sodium thiomalate, ketoprofen, auranofin, aurothioglucose, and tolmetin sodium; antigout agents such as colchicine and allopurinol; anticoagulants such as heparin, heparin sodium, and warfarin sodium; thrombolytic agents such as urokinase, streptokinase, and altoplase; antifibrinolytic agents such as aminocaproic acid; hemorheologic agents such as pentoxifylline; antiplatelet agents such as aspirin, empirin, and ascriptin; anticonvulsants such as valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione; antiparkinson agents such as ethosuximide; antihistamines/antipruritics such as loradatine, hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, and trimprazine tartrate; agents useful for calcium regulation such as calcitonin and parathyroid hormone; antibacterial agents such as amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate; antiviral agents such as interferon gamma, zidovudine, amantadine hydrochloride, ribavirin, and acyclovir; antimicrobials such as cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, erythromycin ethylsuccinate, erythromycin, erythromycin estolate, erythromycin lactobionate, erythromycin siearate, erythromycin ethylsuccinate, tetracycline hydrochloride, doxycycline hyclate, and minocycline hydrochloride; anti-infectives such as GM-CSF; bronchodialators such as epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents, aminophylline, dyphylline, metaproterenol sulfate, aminophylline, mast cell stabilizers, flurisolidebeclomethasone dipropionate, beclomethasone dipropionate monohydrate, salbutamol, beclomethasone dipropionate, ipratropium bromide, budesonide, ketotifen, salmeterol, xinafoate, terbutaline sulfate, triamcinolone, theophylline, nedocromil sodium, metaproterenol sulfate, albuterol, and flunisolide; hormones such as danazol, testosterone cypionate, fluoxymesterone, ethyltostosterone, testosterone enanihate, methyltestosterone, fluoxymesterone, testosterone cypionate, estradiol, estropipate, conjugated estrogens, methoxyprogesterone acetate, norethindrone acetate, triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and thyroid hormones; hypoglycemic agents such as human insulin, purified beef insulin, purified pork insulin, glyburide, chlorpropamide, glipizide, tolbutamide, and tolazamide; hypolipidemic agents such as clofibrate, dextrothyroxine sodium, probucol, lovastatin, and niacin; and agents useful for erythropoiesis stimulation such as erythropoietin.

Surfactants may be used in the compositions disclosed herein to form or stabilize emulsions, enhance solubility, or for a number of other purposes. While not intending to limit the scope of the invention in any way, one type of useful surfactant is a sorbitan ester. Examples include, but are not limited to, Polysorbate 20, Polysorbate 40, Polysorbate 60, and Polysorbate 80.

While not intending to limit the scope of the invention in any way, another type of useful surfactant is a stearate. Examples include, but are not limited to, glyceryl stearate, isopropyl stearate, polyoxyl stearate, propylene glycol stearate, and sucrose stearate.

While not intending to limit the scope of the invention in any way, another useful surfactant is polyethylene glycol.

While not intending to limit the scope of the invention in any way. Other useful surfactants comprise polyethylene oxide or polypropylene oxide. Examples, include, but are not limited to, polyethylene oxides, polypropylene oxides, polyethylene oxide, polypropylene oxide copolymers, alcohol ethoxylates, and alkylphenol ethoxylates.

While not intending to limit the scope of the invention in any way, another useful type of surfactant is alkyl glycosides.

While not intending to limit the scope of the invention in any way, another useful type of surfactant is alkyl polyglycosides

While not intending to limit the scope of the invention in any way, another useful type of surfactant is a fatty alcohol.

While not intending to limit the scope of the invention in any way, another useful type of surfactant is cellulose derivatives, including, but not limited to, hydroxypropylmethyl cellulose (HPMC) and carboxymethyl cellulose (CMC).

While not intending to limit the scope of the invention in any way, another useful type of surfactant is polyacrylic acids, including, but not limited to, Carbomers.

While not intending to limit the scope of the invention in any way, another useful type of surfactant is phospholipids, including, but not limited to, phosphatidyl chloline and phosphatidyl serine.

For emulsions, an oil is used. Any type of oil may be used which is appropriate for the intented use. Vegetable oils are particularly useful, including, but not limited to Castor oil, Clove oil, Cassia oil, Cinnamon oil, Almond oil, Corn oil, Arachis oil, Cottonseed oil, Safflower oil, Maize oil, Linseed oil, Rapeseed oil, Soybean oil, Olive oil, Caraway oil, Rosemary oil, Peanut oil, Peppermint oil, Sunflower oil, Eucalpytus oil, Sesame oil, Mineral oil, Coriander oil, Lavender oil, Citronella oil, Juniper oil, Lemon oil, Orange oil, Clary sage oil, Nutmeg oil, Tea tree oil, and the like.

### EXAMPLES

Useful formulations are prepared according to the table below.

| Ingredient | A | B | C | D |
|---|---|---|---|---|
| Castor Oil (% w/v) | 1.25 | 1.25 | 1.25 | 1.25 |
| Polysorbate-80 (PS-80) | 1 | 1 | 1 | 1 |
| (% w/v) | | | | |
| Pemulen TR-2 (% w/v) | 0.1 | 0.1 | 0.1 | 0.1 |
| Glycerin (% w/v) | 1 | 1 | 1 | 1 |
| Boric Acid (% w/v) | 0.6 | 0.6 | 0.6 | 0.6 |
| Mannitol (% w/v) | N/A | N/A | N/A | N/A |
| Purite ® (% w/v) | 0.0075 | 0.0075 | 0.0075 | 0.0075 |
| Cyclosporine (% w/v) | N/A | 0.05 | | |
| Bimatoprost (% w/v) | | | 0.03 | |
| Prednisolone Acetate | | | | 0.1 |
| (% w/v) | | | | |
| Sodium Hydroxide (% w/v) | pH 7.3 | pH 7.3 | pH 7.3 | pH 7.3 |
| Purified Water (% w/v) | Q.s | Q.s | Q.s | Q.s |

If the glycerin of the composition is substituted with sodium chloride, such that the tonicity is maintained, comparison of the glycerin compositions with the sodium chloride compositions would show that the glycerin compositions have improved preservative efficacy.

Composition A is useful for treating dry eye disease and other irritating ocular conditions.

Composition B is useful for treating dry eye.

Composition C is useful for treating glaucoma or ocular hypertension.

Composition D is useful for treating ocular surface inflammation.

These examples are given only to provide direction and guidance in how to make and use the invention, and are not intended to limit the scope of the invention in any way.

What is claimed is:

1. A preservative consisting of a stabilized oxychloro complex and a polyol having from 3 to 6 carbons.

2. The preservative of claim 1 wherein the stabilized oxychloro complex is from about 0.0001% to about 20% of the preservative.

3. A composition comprising a stabilized chlorine dioxide and a polyol having from 3 to 6 carbons.

4. The composition of claim 3 wherein the polyol is glycerin.

5. The composition of claim 3 wherein the stabilized chlorine dioxide is Purite®.

6. The composition of claim 4 wherein the stabilized chlorine dioxide is Purite®.

7. The composition of claim 6 which further comprises a therapeutically active agent.

8. The composition of claim 7 which is isotonic.

9. The composition of claim 8 which is hypotonic.

10. The composition of claim 9 which is hypertonic.

11. The composition of claim 3 which is an emulsion.

12. The composition of claim 3 wherein the weight ratio of stabilized chlorine dioxide to polyol is from about 0.000001 to about 0.2.

13. The composition of claim 6 wherein the therapeutically active agent is bimatoprost.

14. The composition of claim 6 wherein the therapeutically active agent is cyclosporin A.

15. The composition of claim 6 wherein the therapeutically active agent is prednisolone acetate.

16. The composition of claim 6 wherein the therapeutically active agent is brimonidine.

17. The composition of claim 6 wherein the therapeutically active agent is testosterone.
